(19) United States
(12) Patent Application Publication
(10) Pub. No.: US 2025/0208113 A1
(43) Pub. Date: Jun. 26, 2025

(54) MATERIAL MONITORING SYSTEM AND USES THEREOF

(71) Applicants: THE UNIVERSITY OF TOKYO, Tokyo (JP); BEEINVENTOR LIMITED, Taipei (TW); E-FUJI COMPANY LTD., Taipei (TW)

(72) Inventors: Takafumi NOGUCHI, Tokyo (JP); Shigeyuki NISHIJIMA, Fukuoka (JP); Hiroshi TSUKUI, Tokyo (JP); Kin-Yung CHAN, Taipei (TW); Cheng-Hung WANG, New Taipei (TW)

(21) Appl. No.: 18/844,400

(22) PCT Filed: Mar. 7, 2023
(86) PCT No.: PCT/CN2023/080021

§ 371 Date: Sep. 6, 2024

Related U.S. Application Data
(60) Provisional application No. 63/317,090, filed on Mar. 7, 2022.
(60) Provisional application No. 63/328,744, filed on Apr. 8, 2022.

(30) Foreign Application Priority Data
Mar. 7, 2022 (JP) 2022/035337

(51) Int. Cl. G01N 33/38; G01D 21/02; G01K 1/14; G01N 27/04

(52) U.S. Cl. CPC G01N 33/383; G01D 21/02; G01K 1/14; G01N 27/041

(57) ABSTRACT

The present disclosure provides a material monitoring system for casting concrete. The system includes a monitoring device having a body and an integrated sensor, and the monitoring device is an internet of things (IoT) device. The body of the monitoring device is installed with a power supply and a central processing unit. The integrated sensor is used to measure the real-time temperature and real-time electrical resistance of concrete. Further, the integrated sensor is connected to the bottom surface of the body, and the sensor, the power supply, and the central processing unit are electrically connected. The integrated sensor is immersed in the concrete while the body is situated on the surface of the concrete when the monitoring device is used.

### CROSS-REFERENCE TO RELATED APPLICATIONS

This application claims priority of Japan Patent Application No. 2020035337, filed on Mar. 8, 2022, U.S. Provisional Application 63/317,090, filed on Mar. 7, 2022, and U.S. Provisional Application 63/328,744, filed on Apr. 8, 2022, the content of which is incorporated herein in its entirety by reference.

### FIELD OF THE INVENTION

The present invention relates to a monitoring system. In particular, the invention relates to a system for determining and monitoring concrete strength and further using the system to cast concrete. The present invention further provides a method of using the system to cast concrete.

### BACKGROUND OF THE INVENTION

The prior art teaches a device that can not only measure the surface temperature of concrete and collect data to decide whether to remove the formwork but also can ensure that the device does not remain in the concrete after removing the formwork.

The prior art also teaches a device can precisely measure an effective concrete age by adding a temperature sensor. Generally, a formwork is detached from a concrete building unit 28 days after casting the concrete into the formwork. However, the device is configured to the formwork. Thus, the device cannot stay in the concrete to measure whether the concrete meets a desired condition after the formwork is detached from the concrete.

The prior art further teaches a method of placing Radio-frequency identification (RFID) chips, which permits a sensor on the chips to remain in the concrete to keep measuring the concrete conditions. However, the presence of RFID chips in the concrete may cause serious problems, such as cracks forming in the future, which should be avoided.

The prior art further teaches a device with multiple cables connected to a formwork. However, there are plenty of formworks at a construction site. It is inconvenient, impractical, dangerous, and unrealistic to apply numerous devices with multiple cables to the formworks at the construction site.

To address the forgoing issues, this disclosure aims to provide a method for casting concrete by using a material monitoring system to monitor the status of the concrete. This method can monitor not only the conditions of the concrete in a formwork but also the conditions of the concrete after being detached from the formwork without causing any problems, such as cracks.

### SUMMARY OF THE INVENTION

In view of the above, this disclosure provides a material monitoring system for casting concrete. The system includes a monitoring device having a body and an integrated sensor, and the monitoring device is an internet of things (IoT) device. The body of the monitoring device is installed with a power supply and a central processing unit. The integrated sensor is for measuring a real-time temperature and a real-time electrical resistance of a concrete. Further, the integrated sensor is connected to a bottom surface of the body, and the integrated sensor, the power supply, and the central processing unit are electrically connected. The integrated sensor is immersed in the concrete while the body is situated on a surface of the concrete when the monitoring device is in use.

In one embodiment, the central processing unit is a micro control unit.

In one embodiment, the material monitoring system further includes a remote server communicating with the monitoring device.

In one embodiment, the remote server is a cloud server.

In one embodiment, a predetermined condition is stored in the remote server.

In one embodiment, the predetermined condition includes a temperature, humidity, electrical resistance, strength, or any combination thereof of a concrete member.

In one embodiment, the monitoring device further includes a wireless module.

In one embodiment, the wireless module controls data transmission between the monitoring device and the remote server.

In one embodiment, the wireless module controls data transmission between the monitoring device and a second device. In another embodiment, the second device is an IoT device.

In one embodiment, the integrated sensor is further configured to detect the monitoring device's vibration or movement.

In one embodiment, the integrated sensor is further configured to detect the internal pressure of the concrete.

In one embodiment, the material monitoring system further includes a display unit.

In one embodiment, the integrated sensor is in a conical shape.

In one embodiment, the power supply is a rechargeable battery or a solar panel.

In one embodiment, the monitoring device further includes a memory for storing data received from the integrated sensor.

In one embodiment, the integrated sensor further includes a volume sensor.

Additionally, the present disclosure provides a method for casting concrete with a predetermined strength using the material monitoring system mentioned above. The method includes the following steps: (a) pouring a concrete into the space defined by a formwork, wherein the concrete is in a liquid form; (b) determining a real-time volume of the concrete in the space by the integrated sensor of the monitoring device in the material monitoring system; (c) measuring a real-time temperature of the concrete by the integrated sensor; (d) determining whether the real-time temperature of the concrete meets a first predetermined condition; (e) removing the formwork with the integrated sensor remaining inside the concrete; (f) measuring a real-time electrical resistance of the concrete by the integrated sensor; (g) determining whether a real-time humidity of the concrete meets a second predetermined condition, wherein the real-time humidity is calculated based on the real-time electrical resistance; and (h) removing the monitoring device from the concrete. Generally, the formwork and the material monitoring system are set up before step (a).

In one embodiment, the monitoring device is configured with the formwork, and the integrated sensor is located in the space before the pouring of the concrete.

In one embodiment, step (b) further includes stopping the pouring of the concrete by a signal generated by the material monitoring system if the integrated sensor is immersed in the concrete.

In one embodiment, the monitoring device is placed in the space in a manner to allow free movement before the pouring of the concrete.

In one embodiment, during the pouring of the concrete, the integrated sensor becomes immersed in the concrete while the body is situated on a surface of the concrete.

In one embodiment, the integrated sensor is further configured to detect a vibration or a movement of the monitoring device.

In one embodiment, the real-time volume of the concrete is calculated based on the movement of the monitoring device and a pre-set data.

In one embodiment, step (b) further includes stopping the pouring of the concrete by a signal generated by the material monitoring system if the real-time volume reaches a predetermined volume.

In one embodiment, the method further includes a step (i) of filling a cavity in the concrete caused by the integrated sensor.

In one embodiment, step (e) is implemented if the first predetermined condition is met in step (d); otherwise, steps (c) and (d) are repeated.

In one embodiment, step (h) is implemented if the second predetermined condition is met in step (g); otherwise, steps (f) and (g) are repeated.

In one embodiment, step (g) further includes a step of determining whether the concrete possesses a predetermined strength.

In one embodiment, the material monitoring system further includes a remote server for storing the first and second predetermined conditions.

In one embodiment, the monitoring device further includes a wireless module.

In one embodiment, the monitoring device further includes a memory for storing a data received from the integrated sensor.

### BRIEF DESCRIPTION OF DRAWINGS

FIG. 1 schematically illustrates an embodiment of a material monitoring system of the present disclosure.

FIG. 2 is an embodiment of a system configuration diagram of the material monitoring system.

FIG. 3 is an illustrative flow chart of a concrete casting process of the present disclosure.

FIGS. 4A to 4F illustrate one embodiment of using the material monitoring system of the present disclosure to cast concrete.

FIGS. 5A to 5C illustrate another embodiment of using the material monitoring system of the present disclosure to cast concrete.

FIGS. 6A and 6B are sectional views of other embodiments of the material monitoring system.

### DETAILED DESCRIPTION OF THE INVENTION

The making and using of the embodiments of the disclosure are discussed in detail below. It should be appreciated, however, that the embodiments provide many applicable inventive concepts that can be embodied in a wide variety of specific contexts. The specific embodiments discussed are merely illustrative of specific ways to make and use the embodiments and do not limit the scope of the disclosure.

Reference numerals are used to designate elements throughout the various views and illustrative embodiments. Reference will now be made in detail to exemplary embodiments illustrated in the accompanying drawings. The same reference numbers are used in the drawings and the description wherever possible to refer to the same or like parts. The drawings may exaggerate the shape and thickness for clarity and convenience. This description will be directed in particular to elements forming part of, or cooperating more directly with, an apparatus in accordance with the present disclosure. It is to be understood that elements not specifically shown or described may take various forms. Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. It should be appreciated that the following figures are not drawn to scale; rather, these figures are merely intended for illustration.

The drawings use reference numbers to designate similar elements throughout the various views, and illustrative embodiments of the present disclosure are shown and described. The figures are not necessarily drawn to scale, and in some instances, the drawings have been exaggerated and/or simplified in places for illustrative purposes. One ordinary skill in the art will appreciate the many possible applications and variations of the present disclosure based on the following illustrative embodiments.

### Definition

It will be understood that when an element is referred to as being “on” another element, it can be directly on the other element, or intervening elements may be present. In contrast, when an element is referred to as being “directly on” another element, no intervening elements are present.

It will be understood that singular forms “a,” “an,” and “the” are intended to include the plural forms as well unless the context clearly indicates otherwise. Furthermore, relative terms, such as “bottom” and “top,” may be used herein to describe one element's relationship to other elements, as illustrated in the Figures.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

FIG. 1 schematically illustrates an embodiment of a material monitoring system 1 of the present disclosure. FIG. 1 shows that material monitoring system 1 includes a monitoring device 11 with an integrated sensor 111 and a body 113, and the monitoring device 11 is an internet of things (IoT) device. A central processing unit 1131 and a power supply 1133 are configured inside the body 113. In one embodiment, the central processing unit 1131 is preferably a micro control unit. The power supply 1133 can be a battery to provide sufficient power to run the monitoring device 11. In a preferred embodiment, the power supply 1133 is a rechargeable battery or a solar panel. In one embodiment, the integrated sensor 111 is used to detect temperatures, humidity, pressure, volume, electrical resistance, strength, or acceleration of motion of a concrete structure, or the vibration or movement of the monitoring device 11.

Further, the integrated sensor 111, power supply 1133, and central processing unit 1131 are electrically connected. In another embodiment, the material monitoring system 1 includes a remote server 13, another IoT device 15, and a display unit 17. The monitoring device 11 further includes a wireless module (not shown in the figure). The wireless module controls data transmission between the monitoring device 11 and the remote server 13 or between the monitoring device 11 and another IoT device 15 (e.g., a mobile device). Specifically, LoRa and NBIoT for transmitting data to the remote server 13 and Bluetooth for transmitting data to another IoT device 15 are applied. In another embodiment, the remote server 13 is used to store a predetermined condition. In another embodiment, the body 113 further includes a memory (not shown in the figure) inside for storing the data from the integrated sensor or the predetermined condition.

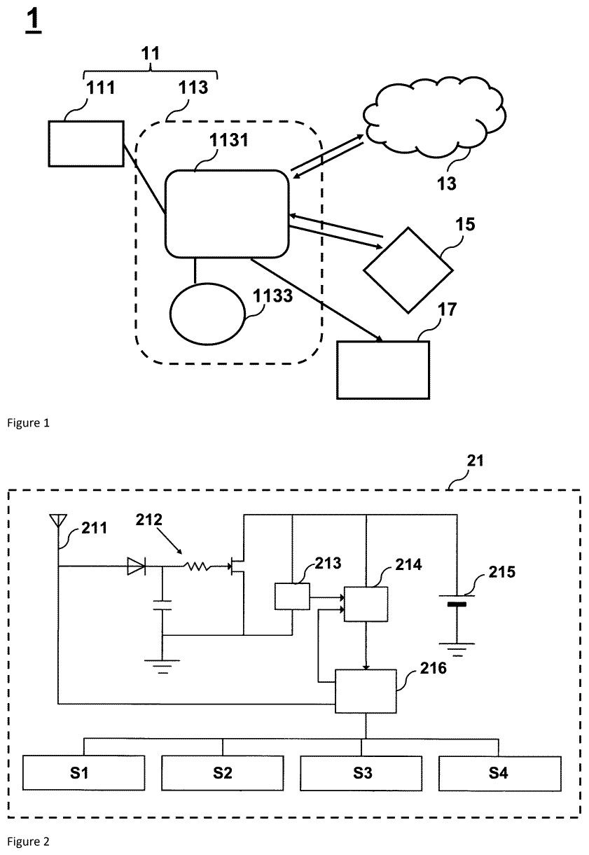

FIG. 2 is an embodiment of a configuration diagram of the monitoring device 21. As FIG. 2 shows, a frequency of 13.56 MHz is used to transmit RFID carriers. However, this is not intended to limit the above frequency; other frequencies may be applied. The monitoring device 21 includes the following elements. An antenna 211 for transmitting and receiving RFID carriers. A voltage application circuit 212 for generating an induced voltage. A field-effect transistor (FET) 213 is switched on, and a regulator 214 is operated when the voltage application circuit generates the induced voltage. The regulator 214 regulates the power from the battery 215 to the micro control unit 216 while operating. The micro control unit 216 receives power and controls the components of the monitoring device 21. The micro control unit 216 is assumed to have a built-in timer.

Further, the monitoring device 21 includes an integrated sensor that may include multiple sensors, such as four sensors (S1 to S4). An orientation sensor S1 detects whether the orientation of the formwork is horizontal or vertical. The orientation sensor S1 may be an accelerometer. The accelerometer can be a mechanical, optical, or semiconductor accelerometer, but the semiconductor accelerometer is preferable because it is inexpensive and easy to handle. In another preferred embodiment, the accelerometer is a 1-axis accelerometer, 2-axis accelerometer, or 3-axis accelerometer. A temperature sensor S2 is for measuring the temperature of the concrete. A volume sensor S3 is for determining whether the concrete reaches a predetermined volume. A humidity sensor S4 is for detecting the humidity of the concrete.

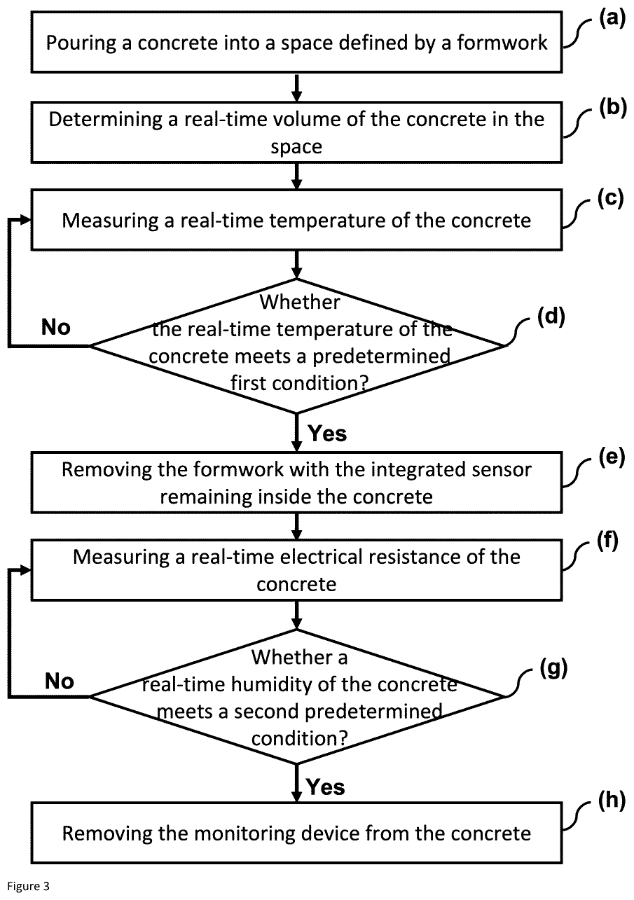

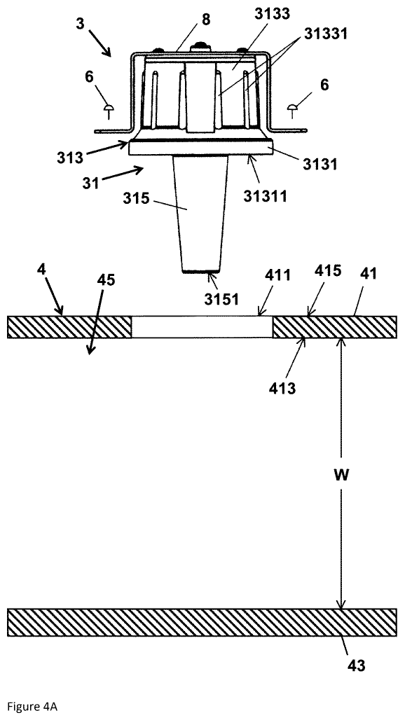

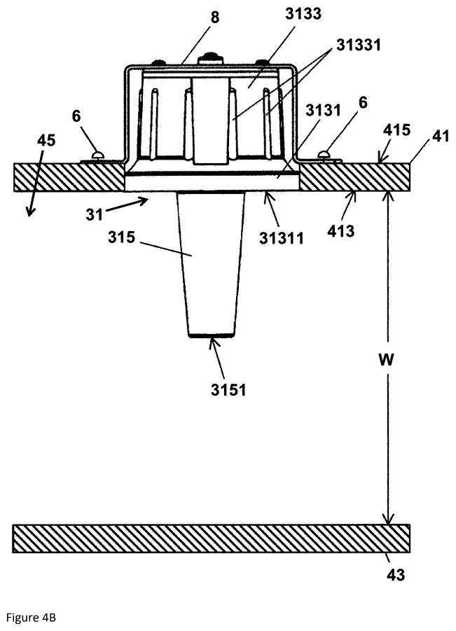

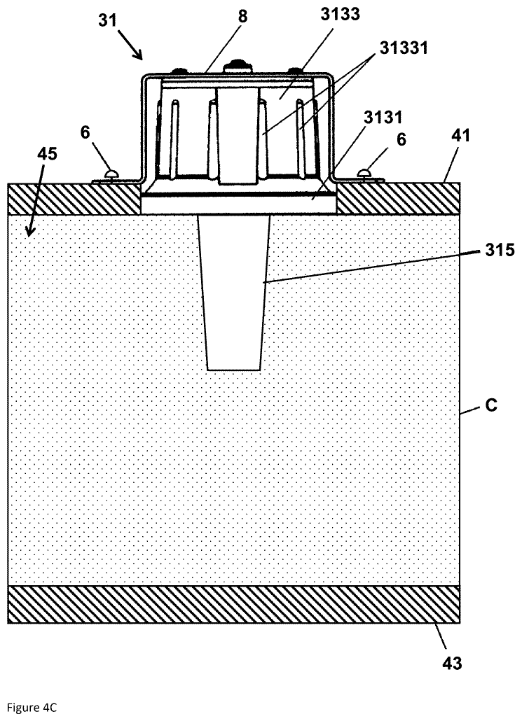

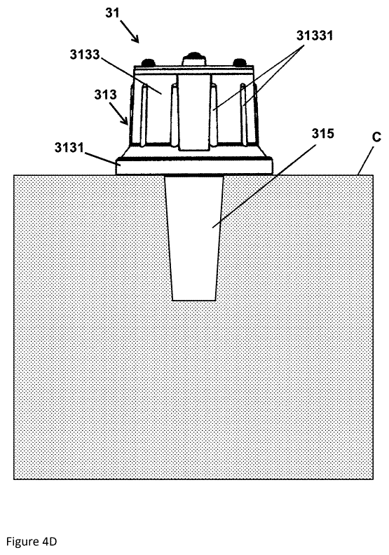

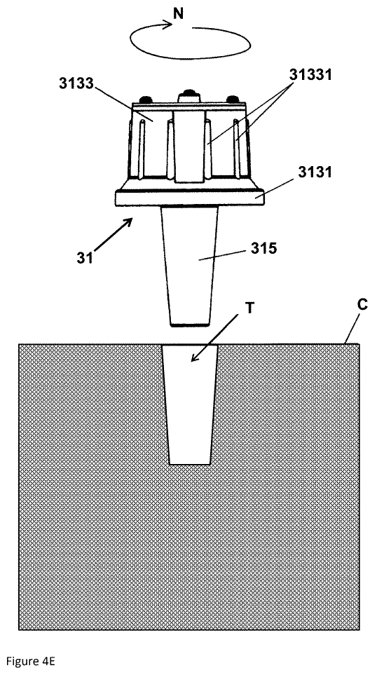

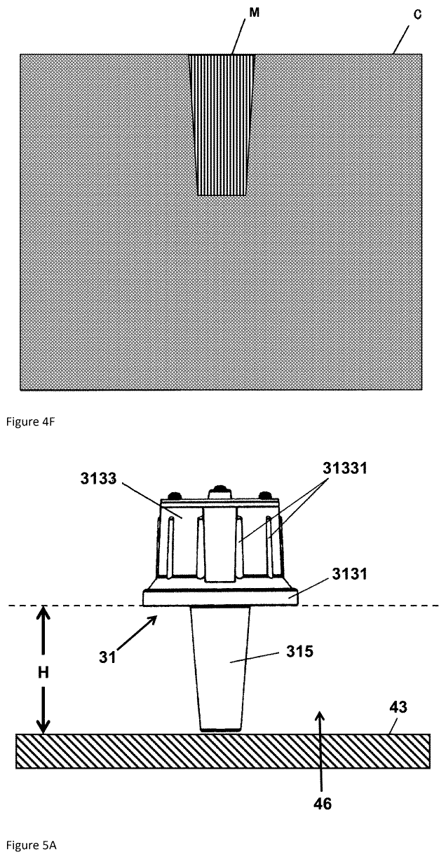

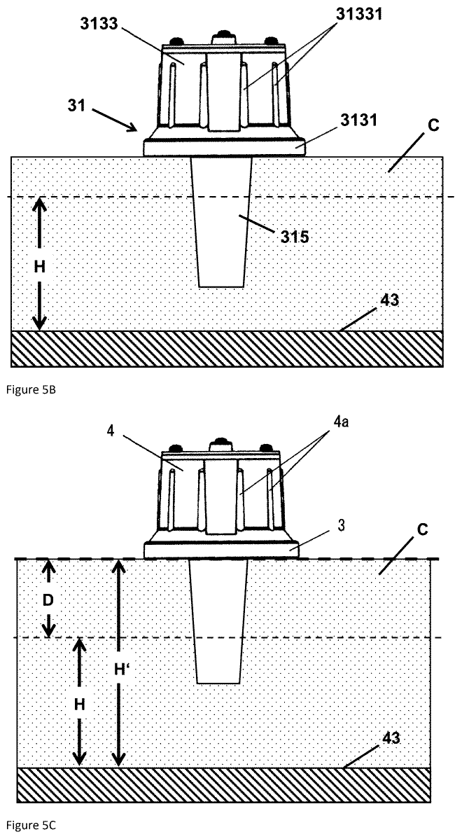

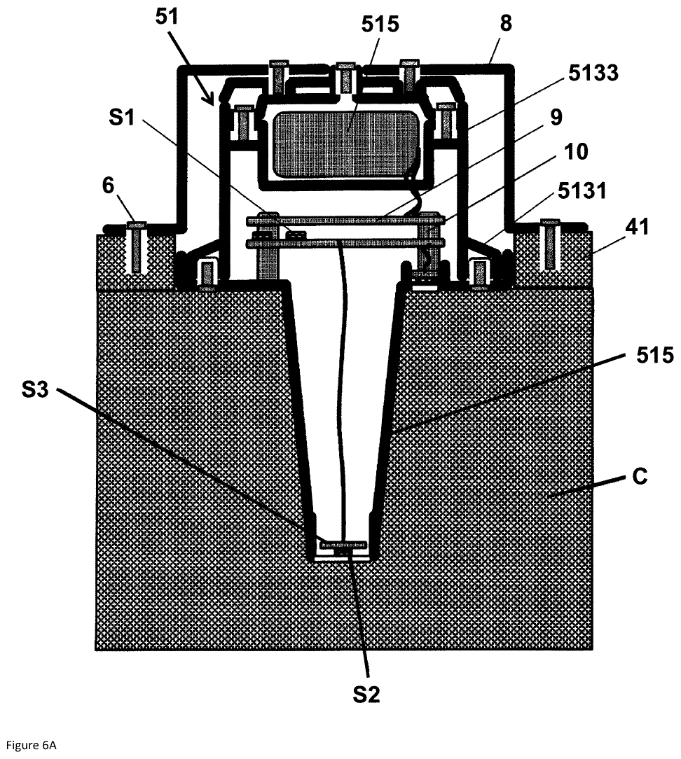

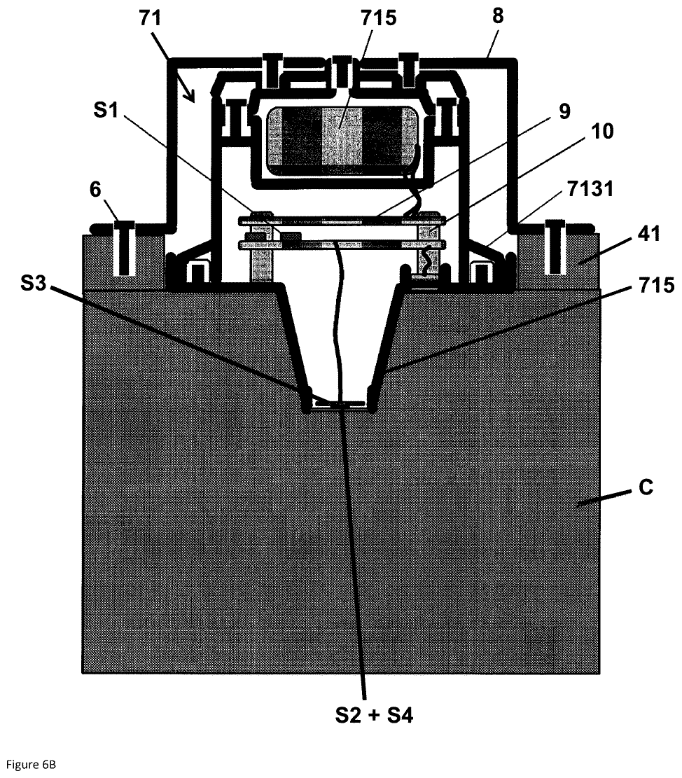

FIG. 3 is an illustrative flow chart of a concrete casting process of the present disclosure. FIGS. 4A to 4F illustrate a process of casting concrete using a material monitoring system 3 in one embodiment, and FIGS. 5A to 6B illustrate a process of casting concrete using a material monitoring system 5 in other embodiments.

Please refer to FIG. 3. The flow chart illustrates the general steps of casting concrete using the material monitoring system. Before step (a), components for casting concrete are prepared, and the material monitoring system and the formwork are set up and installed. The specific configuration status of the system and the formwork will be explained later in the content. Usually, a rebar is incorporated into the formwork. In step (a), concrete in a liquid form is poured into a space defined by the formwork, and then the monitoring device is used to determine a real-time volume of the concrete in the space in step (b). Therefore, the concrete casting begins. The order of step (a) and step (b) may be simultaneous. At this stage, the vibration of the monitoring device may or may not be detected (see FIG. 4C or FIG. 5B) using a vibration sensor. There are no restrictions regarding the type of the concrete C, which may be chosen as appropriate. In one embodiment, step (b) further includes determining whether the real-time volume of the casted concrete reaches a predetermined volume. The material monitoring system generates a signal to stop pouring the concrete if the predetermined volume is met; otherwise, the pouring of the concrete is continued. In step (c), the integrated sensor of the monitoring device measures the real-time temperature of the casted concrete in the formwork.

Next, in step (d), the monitoring device determines, by using the central processing unit (e.g., a micro control unit), whether the real-time temperature of the concrete meets a first predetermined condition. In other words, the material monitoring system will check whether the formwork surrounding the concrete can be removed before the removal in step (e). If the formwork-removal condition is not met, the formwork is maintained in position, and the measurement continues (step (c)). When the formwork-removal condition is satisfied, the formwork is removed, but the monitoring device is still on the concrete. Next, the integrated sensor of the monitoring device measures the concrete's real-time electrical resistance in step (f). In step (g), the monitoring device determines, by using the central processing unit, whether the concrete's real-time humidity meets a second predetermined condition. Further, the real-time humidity is calculated based on the real-time electrical resistance. The first and second predetermined conditions may be stored in a remote server in some embodiments.

In one embodiment, step (g) further includes a step of determining whether the casted concrete possesses a predetermined strength. The material monitoring system generates a signal to remind a user to remove the monitoring device from the concrete if the real-time humidity meets a second predetermined condition; otherwise, the system returns to step (f) to continue measuring the real-time electrical resistance. In some embodiments, the signal is a text message, an alert sound, a light signal, or any combination thereof.

Next, when the user receives the signal from the material monitoring system, the monitoring device is removed from the concrete (step (h)). In one embodiment, the method further includes a step (i) of filling a cavity in the concrete caused by the integrated sensor. Specifically, in step (h), removing the monitoring device from the concrete results in a cavity with the shape of the integrated sensor. The user can fill the cavity with concrete or fillers of other materials for aesthetic or practical reasons. Finally, a concrete building unit with a predetermined strength and a half-life is obtained. In addition, formation of concrete building units using the method mentioned above has the following advantages: (1) it can effectively avoid wasting concrete casting materials to reduce costs; (2) the building units with adequate strength provides the required safety; (3) the concrete casting time can be effectively managed; and (4) the labor required for the application can be reduced and managed more effectively.

FIGS. 4A to 4F illustrate one embodiment of using the material monitoring system of the present disclosure to cast concrete. It is worth knowing that, in this embodiment, the monitoring device of the material monitoring system is configured with the formwork. For example, the monitoring device is fixed on the formwork before casting concrete in step (a) of FIG. 3.

Please refer to FIG. 4A, which illustrates a material monitoring system 3 before being inserted into a formwork 4 (i.e., before step (a) in FIG. 3). As shown in FIG. 4A, the formwork 4 primarily comprises panels 41 and 43, and there is an opening 411 on the panel 41. The opening 411 penetrates in the direction of the thickness of the panel 41. Further, the panels 41 and 43 of the formwork 4 defined a space 45. The space 45 between the panels 41 and 43 is set to align with a dimension W of a concrete structure to be constructed. Further, the following description is based on one embodiment where the panel 41 is at the top and the panel 43 is at the bottom in a vertical relationship to construct a horizontal building unit. However, the person having ordinary skill in the art can easily understand that this disclosure is equally applicable even when the monitoring device 3 is turned sideways, such as when building columns or vertical walls (i.e., when panel 41 is on one side and panel 43 is on the other side), and that such circumstance falls within the scope of protection of present disclosure.

A material monitoring system 3 may have the appearance shown in FIG. 4A and include the following components. As mentioned above, the panel 41 has two surfaces (i.e., a contact surface 413 and a non-contact surface 415), and the non-contact surface 415 is aligned with the outlet of the opening 411. A monitoring device 31 includes a conical portion 315 and a body 313. Further, the body 313 includes a base portion 3131 and a cap portion 3133. The base portion 3131 is preferably disk-shaped. A conical portion 315, which has a tapered conical shape, is to extend from the monitoring device 31 into the space 45 inside the formwork 4. Therefore, as the figure shows, the top end 3151 of the conical portion 315 is a free end located in the space 45. The cap portion 3133, which has a smaller diameter than the base portion 3131, is to be exposed to the outside of the formwork 4. The diameter of the cap portion 3133 is made smaller to facilitate an attachment to and removal from the opening 411 of the panel 41. The surface of cap portion 3133 is preferably a rough surface to prevent the operator's hand from slipping during operation, or as shown in FIG. 4A, it is provided with a protruding strip 31331 or other anti-slip elements.

FIG. 4B illustrates the material monitoring system 3 configured with the formwork 4. Please refer to FIGS. 4A and 4B, after fitting the monitoring device 31 into the opening 411, the monitoring device 31 is fixed to the formwork 4 with screws 6 securing opposing ends of a securing instrument (e.g., the case 8 connected to the top of the cap portion 3133) to the panel 41. Further, when the monitoring device 31 is coupled with the formwork 4 and the case 8 with the screws 6, the bottom surface 31311 of the base portion 3131 will form a continuous plane with the nearby contact surface 413 of the panel 41. From the above description, it is clear that the disclosure of FIGS. 4A and 4B correspond to the set-up step before step (a) in FIG. 3.

FIG. 4C illustrates the space 45 of the formwork 4 being filled with concrete C after the monitoring device is coupled to the panel 41. Further, the disclosure of FIG. 4C corresponds to steps (a) to (d) in FIG. 3. Briefly, the concrete is poured continuously into the space 45 until the space 45 is filled up or the volume of the poured concrete reaches a predetermined volume, and the pouring of the concrete C may be stopped by a signal generated by the monitoring device. In one embodiment, the predetermined volume refers to the volume at which the conical portion 315 is entirely surrounded by the concrete C (i.e., when the conical portion 315 is wholly immersed in the concrete C). In another embodiment, the predetermined volume refers to the volume at which the conical portion 315 is partially surrounded by the concrete C, e.g., only the top end 3151 of the conical portion 315 is fully submerged in the concrete C (not shown in FIG. 4C).

Further, the coupling between the monitoring device 31 and the formwork 4 is maintained until the formwork 4 is removed (i.e., until the liquid-form concrete has hardened and contracted sufficiently from its soft state). During the period, the monitoring device 31 is secured to the panel 41 via the case 8 so that the conical portion 315 becomes buried inside the concrete C. Once the monitoring device 31 is in the state shown in FIG. 4C, the properties of the concrete C (e.g., temperatures) can be measured as shown in step (c) of FIG. 3. More specifically, please refer to FIG. 2, data collected by each of the sensors S1 to S4 can be transmitted from an antenna 211 to a second IoT device (not shown in the figure, e.g., a smartphone, personal computer, etc.). The second IoT device can store the data or further transmit it to a remote server at a predetermined site through any other communication method known to those skilled in the art. During this period, the micro control unit 216 (see FIG. 2) checks whether the formwork-removal condition is met. If the formwork-removal condition is not met, the formwork 4 stays in position, and the measurement continues (see step (c) in FIG. 3).

As described above for step (d) in FIG. 3, the formwork 4 is removed with the monitoring device 31 remaining on the concrete C. More specifically, as shown in FIG. 4D, when the concrete C hardens to a certain level, the case 8, screws 6, and panels 41 and 43 (i.e., formwork 4) are removed. The monitoring device 31 has only the conical portion 315 inserted in the hardened concrete C. At this stage, an integrated sensor in the conical portion 315 of monitoring device 31 can continue to measure the real-time electrical resistance of the concrete C (step (f) in FIG. 3). It is worth knowing that this applies not only when the panel 41 is at the top and the panel 43 is at the bottom, but also when the panel 41 is on one side and the panel 43 is on the other side. In fact, it should be noted that this disclosure also provides that when concrete C is hardened and contracted, the conical portion 315 is strongly connected to the concrete C and cannot be easily detached. As shown in steps (f) and (g) of FIG. 3, the measurement and communication by the material monitoring system 3 continue until the strength of concrete C reaches a predetermined condition. Therefore, this embodiment ensures the measurement of the humidity of the concrete and the communication of the humidity data, even after the formwork is dismantled.

After the concrete C possesses the predetermined strength, as shown in FIG. 4E, the user can grab the cap portion 3133 by hand and rotate the cap portion 3133 in the direction N of the arrow, that is, to separate the conical portion 315 from the concrete C. Usually, around 28 days after concrete casting, the concrete becomes hard enough, and the formwork can be removed. Still, the exact time required for the concrete C to gain the predetermined strength depends on the site conditions. To easily separate the conical portion 315 from the cured concrete, the surface of the conical portion 315 may be lubricated or provided with a release layer. Therefore, no components of the material monitoring system 3 remain in the cured concrete C after the monitoring device 31 is removed.

FIG. 4F is a schematic illustration of the cavity T caused by the monitoring device 31 in the concrete C and further filled with a filler M. As described above for step (i) in FIG. 3, removing the monitoring device 31 from the concrete C would leave a cavity with the shape of the integrated sensor (e.g., in a conical shape). The user can fill the cavity with concrete C or a filler M of other materials for aesthetic or practical reasons.

As shown in FIGS. 5A to 5C, the present disclosure provides another embodiment of using the monitoring device 31 of the material monitoring system 3 to cast concrete. It is worth knowing that the method (FIG. 3) of casting concrete building units using the material monitoring system 3 in this embodiment differs only in (1) the set-up step before step (a) and (2) step (b), compared to the embodiment described in FIGS. 4D-4F. The other steps in the concrete casting process and the functioning of the material monitoring system 3 are essentially identical. Therefore, the same steps and functions are not repeated in the following. In this embodiment, the monitoring device 31 does not couple with the formwork 4 but is put on the panel 43, allowing the monitoring device 31 to move freely. Please refer to FIG. 5A, the monitoring device 31 of the material monitoring system 3 is placed and stands on the panel 43. The components of the monitoring device 31 are as described in the FIG. 4A. However, only the panel 43 is used in this embodiment; therefore, the space 46 defined by the panel 43 is not closed but has an open end (e.g., a box with an opening). This formwork is usually used to cast a floor, for example. When the monitoring device 31 stands on the panel 43, the integrated sensor in the monitoring device 31 measures the distance (i.e., height H) between it and the panel 43 and stores the unique default value of height H. In another embodiment, the integrated sensor is in the base portion 3131 or the cap portion 3133. In another embodiment, the integrated sensor is in a conical portion 315.

Please refer to FIG. 5B, since the specific gravity (or relative density) of the monitoring device 31 is lower than that of the concrete C, the monitoring device 31 can float on the surface of the concrete C after the concrete C is poured into the formwork 4. More specifically, the base portion 3131 and the cap portion 3133 of the monitoring device 31 is situated on the concrete C. In contrast, the conical portion 315 is immersed in the concrete C. FIG. 5C discloses that the monitoring device 31 would move upward by a distance D when the concrete C is filled to a certain amount so that the monitoring device 31 can measure the real-time distance D and estimate the height H′ (i.e., the distance D plus the default height H) of the concrete C poured above the panel 46. Further, the monitoring device 31, by using the distance H′ and predetermined data (e.g., the base area of the formwork 4), can calculate the volume of the concrete C that has been poured in real-time and determine if the concrete C has reached the desired volume and whether the pouring of concrete should be stopped. In another embodiment, the predetermined data is stored in the remote server. In another embodiment, the predetermined data is stored in the memory of the monitoring device 31.

Described below are two embodiments regarding the length of the conical portion 315.

Please refer to FIG. 6A. In this embodiment, the length of the conical portion 515 extending from the bottom of the base portion 5131 is longer than the height of the cap portion 5133. The cap portion 5133 is equipped with an integrated circuit board 9, and the circuitry and related electronic components disclosed in FIG. 2 are mounted on the integrated circuit board 9. The cap portion 5133 also has pillars 10 to support the integrated circuit board 9 in the cap portion 5133. The monitoring device 51 disclosed in this embodiment (FIG. 6A) is suitable for conducting measurement in the deeper layers of concrete C. In deeper layers, the humidity sensor S4 can be omitted. In another embodiment (please refer to FIG. 6B), the length of the conical portion 615 extending from the bottom of the base portion 6131 is shorter than the height of the cap portion 6133. Therefore, it is preferable to install the humidity sensor S4 at approximately the same position as the temperature sensor S2. The primary reason is that the surface layer of concrete tends to dry out more quickly. After removing the formwork, it is preferable to measure the relative humidity with the humidity sensor S4. Therefore, this embodiment (FIG. 6B) is suitable for conducting measurements in the surface layer of the concrete C.

This disclosure enables continuous measurement of the conditions of the concrete, not only while a formwork is attached to the concrete but also after the formwork is detached from the concrete. This is because a conical portion of the monitoring device remains gripped by the hardened and contracted concrete until the monitoring device is removed after the concrete reaches a predetermined strength. In particular, the measurement can be carried out without any trouble by the material monitoring system, even when many formworks are used at a construction site. After the monitoring device is removed from the concrete, no components of the material monitoring system remain inside the concrete, preventing cracks or other defects resulting from the presence of the monitoring device.

### BRIEF DESCRIPTION OF SYMBOLS

Material monitoring system 1, 3
Monitoring device 11, 21, 31, 51, 71
Integrated sensor 111, 311
Body 113, 313
Central processing unit 1131
Power supply 1133
Remote server 13
IoT device 15
Display unit 17
Antenna 211
Voltage application circuit 212
Field-effect transistor (FET) 213
Regulator 214
Battery 215, 515, 715
Micro control unit 216
Conical portion 315, 615
Top end 3151
Base portion 3131, 5131, 7131
Bottom surface 31311
Cap portion 3133, 5133, 6133
Protruding strip 31331
Formwork 4
Panel 41, 43
Opening 411
Contact surface 413
Non-contact surface 415
Space 45, 46
Screw 6
Case 8
Integrated circuit board 9
Pillar 10
Concrete C
Distance D
Height H, H′
Filler M
Direction N
Orientation sensor S1
Temperature sensor S2
Volume sensor S3
Humidity sensor S4
Cavity T
Dimension W

1. A material monitoring system, comprising:
a monitoring device, wherein the monitoring device is an internet of things (IoT) device, including:
a body installed with a power supply and a central processing unit; and
an integrated sensor for measuring a real-time temperature and a real-time electrical resistance of a concrete, wherein the integrated sensor is connected to a bottom surface of the body, and the integrated sensor, the power supply, and the central processing unit are electrically connected;
wherein the integrated sensor is immersed in the concrete while the body is situated on a surface of the concrete when the monitoring device is in use.

2. The material monitoring system of claim 1, further comprises a remote server communicating with the monitoring device.

3. The material monitoring system of claim 2, wherein a predetermined condition is stored in the remote server.

4. The material monitoring system of claim 3, wherein the predetermined condition comprises a temperature, a humidity, an electrical resistance, a strength, or any combination thereof of a concrete member.

5. The material monitoring system of claim 2, wherein the monitoring device further comprises a wireless module.

6. The material monitoring system of claim 5, wherein the wireless module controls a data transmission between the monitoring device and the remote server.

7. The material monitoring system of claim 5, wherein the wireless module controls a data transmission between the monitoring device and a second IoT device.

8. The material monitoring system of claim 1, wherein the integrated sensor is further configured to detect a vibration or a movement of the monitoring device.

9. The material monitoring system of claim 1, wherein the integrated sensor is further configured to detect an internal pressure of the concrete.

10. The material monitoring system of claim 1, wherein the material monitoring system further comprises a display unit.

11. The material monitoring system of claim 1, wherein the integrated sensor is in a conical shape.

12. The material monitoring system of claim 1, wherein the power supply is a rechargeable battery or a solar panel.

13. The material monitoring system of claim 1, wherein the monitoring device further comprises a memory for storing a data received from the integrated sensor.

14. The material monitoring system of claim 1, wherein the integrated sensor further comprises a volume sensor.

15. A method for casting a concrete with a predetermined strength by using the material monitoring system of claim 1, comprising:
(a) pouring a concrete into a space defined by a formwork, wherein the concrete is in a liquid form;
(b) determining a real-time volume of the concrete in the space by the integrated sensor of the monitoring device in the material monitoring system;
(c) measuring a real-time temperature of the concrete by the integrated sensor;
(d) determining whether the real-time temperature of the concrete meets a first predetermined condition;
(e) removing the formwork with the integrated sensor remaining inside the concrete;
(f) measuring a real-time electrical resistance of the concrete by the integrated sensor;
(g) determining whether a real-time humidity of the concrete meets a second predetermined condition, wherein the real-time humidity is calculated based on the real-time electrical resistance; and
(h) removing the monitoring device from the concrete.

16. The method of claim 15, wherein the monitoring device is configured with the formwork, and the integrated sensor is located in the space before the pouring of the concrete.

17. The method of claim 16, wherein step (b) further comprises stopping the pouring of the concrete by a signal generated by the material monitoring system if the integrated sensor is immersed in the concrete.

18. The method of claim 15, wherein the monitoring device is placed in the space in a manner to allow free movement before the pouring of the concrete.

19. The method of claim 18, wherein during the pouring of the concrete, the integrated sensor becomes immersed in the concrete while the body is situated on a surface of the concrete.

20. The method of claim 18, wherein the integrated sensor is further configured to detect a vibration or a movement of the monitoring device.

21. The method of claim 20, wherein the real-time volume is calculated based on the movement of the monitoring device and a pre-set data.

22. The method of claim 18, wherein step (b) further comprises stopping the pouring of the concrete by a signal generated by the material monitoring system if the real-time volume reaches a predetermined volume.

23. The method of claim 15, further comprises step (i) of filling a cavity in the concrete caused by the integrated sensor.

24. The method of claim 15, wherein step (e) is implemented if the first predetermined condition is met in step (d); otherwise steps (c) and (d) are repeated.

25. The method of claim 15, wherein step (h) is implemented if the second predetermined condition is met in step (g); otherwise steps (f) and (g) are repeated.

26. The method of claim 15, wherein step (g) further comprises a step of determining whether the concrete possesses a predetermined strength.

27. The method of claim 15, wherein the material monitoring system further comprises a remote server for storing the first and second predetermined conditions.

28. The method of claim 27, wherein the monitoring device further comprises a wireless module.

29. The method of claim 15, wherein the monitoring device further comprises a memory for storing a data received from the integrated sensor.
